(19) United States
(12) Patent Application Publication
(10) Pub. No.: US 2025/0331912 A1
(43) Pub. Date: Oct. 30, 2025

(54) COMBINATION ULTRASONIC AND PLASMA INSTRUMENT

(71) Applicant: Covidien LP, Mansfield, MA (US)

(72) Inventors: Matthew S. COWLEY, Boulder, CO (US); Michael B. LYONS, Boulder, CO (US); David J. VAN TOL, Boulder, CO (US); Richard L. CROFT, Boulder, CO (US)

(73) Assignee: Covidien LP, Mansfield, MA (US)

(21) Appl. No.: 18/865,571

(22) PCT Filed: May 12, 2023
(86) PCT No.: PCT/IB2023/054937

§ 371 Date: Nov. 13, 2024

Related U.S. Application Data
(60) Provisional application No. 63/342,197, filed on May 16, 2022.

(51) Int. Cl. A61B 18/14; A61B 18/00; A61B 18/12

(52) U.S. Cl. CPC A61B 18/14; A61B 18/1206; A61B 2018/00077; A61B 2018/00083; A61B 2018/00589; A61B 2018/00601; A61B 2018/00994; A61B 2018/1253; A61B 2018/1412

(57) ABSTRACT

A combination ultrasonic and electrosurgical surgical instrument includes a housing having an ultrasonic transducer disposed within the housing and a wave guide configured to support an ultrasonic blade operably coupled to the ultrasonic transducer. The ultrasonic blade is configured to receive ultrasonic energy produced by the ultrasonic transducer. The ultrasonic blade is tapered along both a vertical axis and a horizontal axis defined therealong and defines an elongated edge on an upper surface thereof terminating at a spatula-like distal end. The elongated edge and side of the spatula-like distal end are coated with an electrically conductive material and are both adapted to connect to a source of electrosurgical energy such that activation of the electrosurgical energy source and the ultrasonic transducer treat tissue with both electrosurgical energy and ultrasonic energy.

### FIELD

The present disclosure relates to energy based surgical instruments and, more particularly, to surgical instruments and systems incorporating both ultrasonic and/or electrosurgical functionality to facilitate energy-based tissue treatment.

### BACKGROUND

Ultrasonic surgical instruments and systems utilize ultrasonic energy, i.e., ultrasonic vibrations, to treat tissue. More specifically, ultrasonic surgical instruments and systems utilize mechanical vibration energy transmitted at ultrasonic frequencies to treat tissue. An ultrasonic surgical device may include, for example, an ultrasonic blade and a clamp mechanism to enable clamping of tissue against the blade. Ultrasonic energy transmitted to the blade causes the blade to vibrate at very high frequencies, which allows for heating tissue to treat tissue clamped against or otherwise in contact with the blade.

Electrosurgical instruments and systems conduct Radio Frequency (RF) energy through tissue to treat tissue. An electrosurgical instrument or system may be configured to conduct bipolar RF energy between oppositely charged electrodes and through tissue, e.g., tissue clamped between the electrodes or otherwise in contact therewith, to treat tissue. Alternatively or additionally, an electrosurgical instrument or system may be configured to deliver monopolar RF energy from an active electrode to tissue in contact with the electrode, with the energy returning via a remote return electrode device to complete the circuit.

### SUMMARY

As used herein, the term “distal” refers to the portion that is described which is further from an operator (whether a human surgeon or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, and/or other variations, up to and including plus or minus 10 percent. Further, any or all of the aspects described herein, to the extent consistent, may be used in conjunction with any or all of the other aspects described herein.

Provided in accordance with aspects of the present disclosure is a combination ultrasonic and electrosurgical surgical instrument which includes a housing having an ultrasonic transducer disposed within the housing and a wave guide configured to support an ultrasonic blade operably coupled to the ultrasonic transducer. The ultrasonic blade is configured to receive ultrasonic energy produced by the ultrasonic transducer. The ultrasonic blade is tapered along both a vertical axis and a horizontal axis defined therealong and defines an elongated edge on an upper surface thereof terminating at a spatula-like distal end. The elongated edge and side of the spatula-like distal end are coated with an electrically conductive material and are both adapted to connect to a source of electrosurgical energy such that activation of the electrosurgical energy source and the ultrasonic transducer treat tissue with both electrosurgical energy and ultrasonic energy.

In aspects according to the present disclosure, the ultrasonic transducer is separably activable relative to the source of electrosurgical energy.

In aspects according to the present disclosure, the ultrasonic transducer is energized by the source of electrosurgical energy.

In aspects according to the present disclosure, the electrically conductive material on the elongated edge and the side of the spatula-like distal end are separated by an insulated material and are independently activatable by a switch.

In aspects according to the present disclosure, an insulating sheath is disposed between the electrically conductive material on the elongated edge and the side of the spatula-like distal end.

In aspects according to the present disclosure, ultrasonic and monopolar energy are simultaneously provided to the elongated edge to quickly and finely dissect tissue.

In aspects according to the present disclosure, monopolar energy is provided to the elongated edge to dissect tissue.

In aspects according to the present disclosure, the side surface of the spatula-like distal end is used to simultaneously treat tissue with ultrasonic energy and monopolar energy to coagulate tissue.

In aspects according to the present disclosure, the side surface of the spatula-like distal end is used to treat tissue with ultrasonic energy to coagulate tissue.

In aspects according to the present disclosure, the waveguide is coated with an insulative material selected from the group consisting of glass, ceramic and polymer.

Provided in accordance with aspects of the present disclosure is a combination ultrasonic and electrosurgical surgical instrument having a housing including an ultrasonic transducer disposed within the housing a wave guide configured to support an ultrasonic blade operably coupled to the ultrasonic transducer. The ultrasonic blade is configured to receive ultrasonic energy produced by the ultrasonic transducer. The ultrasonic blade is tapered along both a vertical and a horizontal axis defined therealong and defines an elongated edge on an upper surface thereof terminating at a spatula-like distal end. The elongated edge and side of the spatula-like distal end are electrically conductive and are separated by an insulative material. The elongated edge and the spatula-like distal end are adapted to independently connect to a source of electrosurgical energy such that activation of the electrosurgical energy source treats tissue with both monopolar electrosurgical energy and ultrasonic energy.

In aspects according to the present disclosure, the ultrasonic transducer is adapted to connect to a separate source of electrical energy.

In aspects according to the present disclosure, the electrically conductive material on the elongated edge and the side of the spatula-like distal end are independently activatable by a switch.

In aspects according to the present disclosure, the electrically conductive material on the elongated edge and the side of the spatula-like distal end are independently activatable by a switch controlled by an algorithm.

In aspects according to the present disclosure, an insulating sheath is disposed between the electrically conductive material on the elongated edge and the side of the spatula-like distal end.

In aspects according to the present disclosure, both ultrasonic and monopolar energy are simultaneously provided to the elongated edge to quickly and finely dissect tissue.

In aspects according to the present disclosure, monopolar energy is provided to the elongated edge to dissect tissue.

In aspects according to the present disclosure, the side surface of the spatula-like distal end is used to simultaneously treat tissue with ultrasonic energy and monopolar energy to coagulate tissue.

In aspects according to the present disclosure, the side surface of the spatula-like distal end is used to treat tissue with ultrasonic energy to coagulate tissue.

In aspects according to the present disclosure, the waveguide is coated with an insulative material selected from the group consisting of glass, ceramic and polymer.

### BRIEF DESCRIPTION OF THE DRAWINGS

The above and other aspects and features of the present disclosure will become more apparent in light of the following detailed description when taken in conjunction with the accompanying drawings wherein like reference numerals identify similar or identical elements.

FIG. 1 is a side view of a surgical system provided in accordance with the present disclosure including a surgical instrument, a surgical generator, and a return electrode device;

FIG. 2 is perspective view of another surgical system provided in accordance with the present disclosure including a surgical instrument incorporating an ultrasonic generator, electrosurgical generator, and power source therein;

FIG. 3 is a schematic illustration of a robotic surgical system provided in accordance with the present disclosure;

FIG. 4 is a longitudinal, cross-sectional view of a distal end portion of the surgical instrument of FIG. 1;

FIG. 5 is a transverse, cross-sectional view of the end effector assembly of a combination ultrasonic and electrosurgical surgical scalpel for use with the system of FIG. 1; and

FIG. 6A-6C are various enlarged views of a blade of the combination ultrasonic and electrosurgical surgical scalpel FIG. 5.

### DETAILED DESCRIPTION

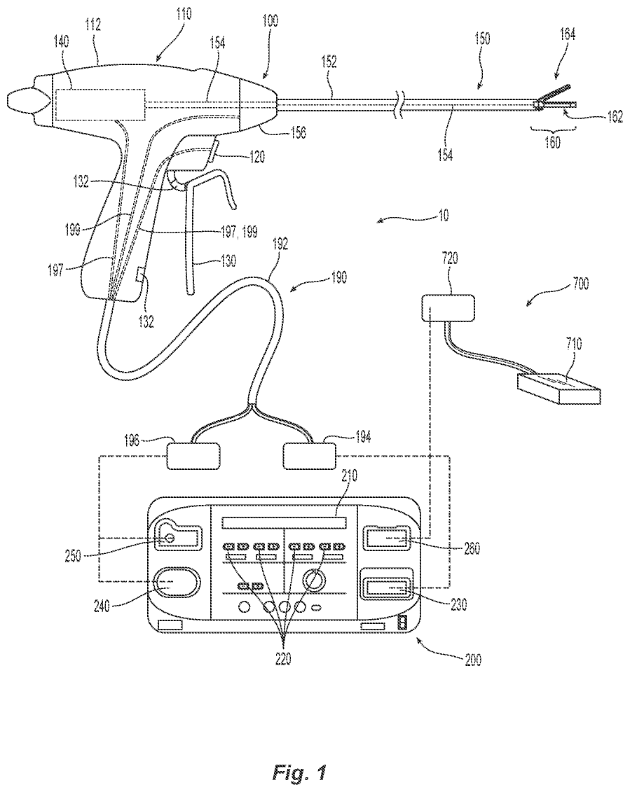

Referring to FIG. 1, a surgical system provided in accordance with aspects of the present disclosure is shown generally identified by reference numeral 10 including a surgical instrument 100, a surgical generator 200, and, in some aspects, a return electrode device 700, e.g., including a return pad 710. Surgical instrument 100 includes a handle assembly 110, an elongated assembly 150 extending distally from handle assembly 110, an end effector assembly 160 disposed at a distal end of elongated assembly 150, and a cable assembly 190 operably coupled with handle assembly 110 and extending therefrom for connection to surgical generator 200.

Surgical generator 200 includes a display 210, a plurality user interface features 220, e.g., buttons, touch screens, switches, etc., an ultrasonic plug port 230, a bipolar electrosurgical plug port 240, and active and return monopolar electrosurgical plug ports 250, 260, respectively. As an alternative to plural dedicated ports 230-260, one or more common ports (not shown) may be configured to act as any two or more of ports 230-260.

Surgical instrument 100 is configured to supply electrosurgical, e.g., Radio Frequency (RF), energy to tissue to treat tissue, e.g., in a monopolar configuration and/or a bipolar configuration, and/or to supply ultrasonic energy to tissue to treat tissue. Surgical generator 200 is configured to produce ultrasonic drive signals for output through ultrasonic plug port 230 to surgical instrument 100 (in aspects where surgical instrument 100 is configured to deliver ultrasonic energy) to activate surgical instrument 100 to supply ultrasonic energy and to provide electrosurgical energy, e.g., RF bipolar energy for output through bipolar electrosurgical plug port 240 and/or RF monopolar energy for output through active monopolar electrosurgical port 250 to surgical instrument 100 (in aspects where surgical instrument 100 is configured to deliver electrosurgical energy) to activate surgical instrument 100 to supply electrosurgical energy. Plug 520 of return electrode device 700 is configured to connect to return monopolar electrosurgical plug port 260 to return monopolar electrosurgical energy from surgical instrument 100 during monopolar electrosurgical use.

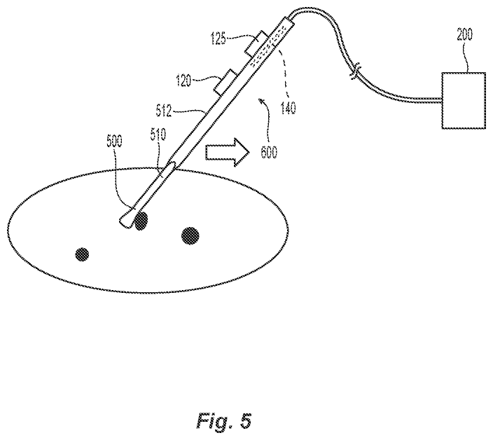

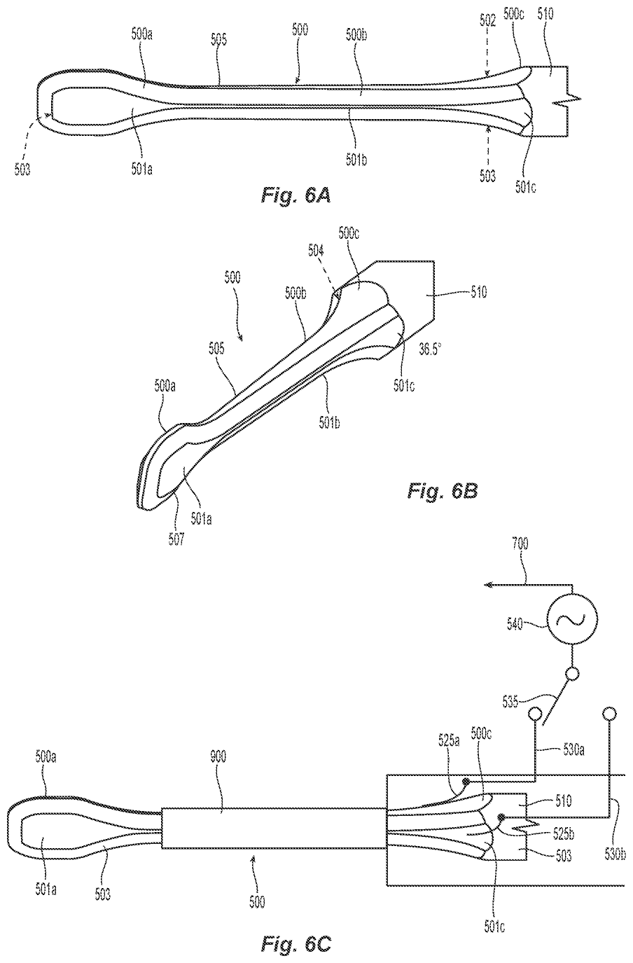

Surgical instrument 100 is generally described herein as an example of one such instrument that the blade 500 of the present disclosure may be configured to work with since surgical instrument 100 utilizes both ultrasonic energy and monopolar energy and may be configured to operably couple to the blade and the various electrical components associated therewith. For example, and as shown in FIGS. 5-6C, a surgical scalpel 600 may be configured to couple with the blade 500 and utilize both ultrasonic and high energy monopolar energy to treat tissue for spot coagulation or fine dissection as described herein. Moreover, other surgical instruments (not shown) may also be adapted to couple with the blade 500 and suit the surgical purposes described herein. As such, surgical instrument 100 will be initially described in sufficient detail for an understanding of the mechanical and electrical operational details of a combination ultrasonic and electrosurgical instrument from a general perspective leaving the details of the blade 500 and the features relating thereto for discussion with reference to FIGS. 5-6C.

Continuing with reference to FIG. 1, handle assembly 110 includes a housing 112, an activation button 120, and a clamp lever 130. Housing 112 is configured to support an ultrasonic transducer 140. Ultrasonic transducer 140 may be permanently engaged within housing 112 or removable therefrom. Ultrasonic transducer 140 includes a piezoelectric stack or other suitable ultrasonic transducer components electrically coupled to surgical generator 200, e.g., via one or more of first electrical lead wires 197, to enable communication of ultrasonic drive signals to ultrasonic transducer 140 to drive ultrasonic transducer 140 to produce ultrasonic vibration energy that is transmitted along a waveguide 154 of elongated assembly 150 to blade 162 of end effector assembly 160 of elongated assembly 150, as detailed below. Feedback and/or control signals may likewise be communicated between ultrasonic transducer 140 and surgical generator 200. Ultrasonic transducer 140, more specifically, may include a stack of piezoelectric elements secured, under pre-compression between proximal and distal end masses or a proximal end mass and an ultrasonic horn with first and second electrodes electrically coupled between piezoelectric elements of the stack of piezoelectric elements to enable energization thereof to produce ultrasonic energy. However, other suitable ultrasonic transducer configurations, including plural transducers and/or non-longitudinal, e.g., torsional, transducers are also contemplated.

An activation button 120 is disposed on housing 112 and coupled to or between ultrasonic transducer 140 and/or surgical generator 200, e.g., via one or more of first electrical lead wires 197, to enable activation of ultrasonic transducer 140 in response to depression of activation button 120. In some configurations, activation button 120 may include an ON/OFF switch. In other configurations, activation button 120 may include multiple actuation switches to enable activation from an OFF state to different states corresponding to different activation settings, e.g., a first state corresponding to a first activation setting (such as a LOW power and/or tissue sealing setting) and a second state corresponding to a second activation setting (such as a HIGH power and/or tissue transection setting). In still other configurations, separate activation buttons may be provided, e.g., a first actuation button for activating a first activation setting and a second activation button for activating a second activation setting. Additional activation buttons, sliders, wheels, etc. are also contemplated to enable control of various different activation settings from housing 112. Other activation buttons may be disposed on the housing 112 for activating other energy modalities, e.g., activation button 125 for activating monopolar energy (FIG. 5).

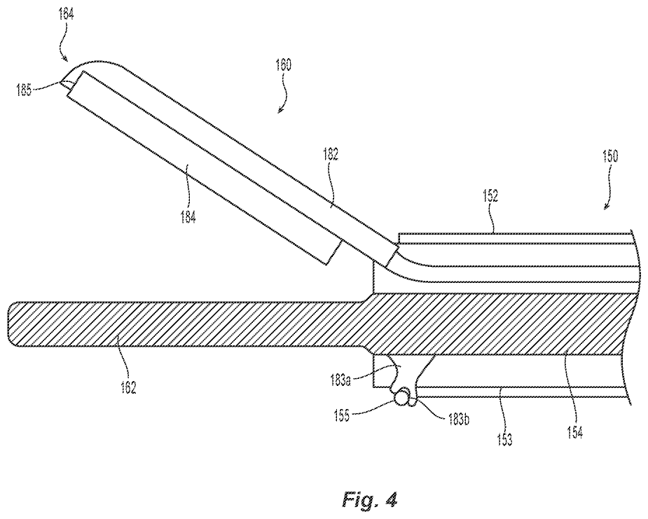

Elongated assembly 150 of surgical instrument 100 includes an outer drive sleeve 152, an inner support sleeve 153 (FIG. 4) disposed within outer drive sleeve 152, a waveguide 510 extending through inner support sleeve 153 (FIG. 4), a drive assembly (not shown), a rotation knob 156, and an end effector assembly 160 including a blade 500 and a jaw member 164. Rotation knob 156 is rotatable in either direction to rotate elongated assembly 150 in either direction relative to handle assembly 110. The drive assembly operably couples a proximal portion of outer drive sleeve 152 to clamp lever 130 of handle assembly 110. A distal portion of outer drive sleeve 152 is operably coupled to jaw member 164 and a distal end of inner support sleeve 153 (FIG. 4) pivotably supports jaw member 164. As such, clamp lever 130 is selectively actuatable, e.g., between an un-actuated position and a fully actuated position, to thereby move outer drive sleeve 152 about inner support sleeve 153 (FIG. 4) to pivot jaw member 164 relative to blade 162 of end effector assembly 160 from an open position towards a closed position for clamping tissue between jaw member 164 and blade 500. The configuration of outer and inner sleeves 152, 153 (FIG. 4) may be reversed, e.g., wherein outer sleeve 152 is the support sleeve and inner sleeve 153 (FIG. 4) is the drive sleeve. Other suitable drive structures as opposed to a sleeve are also contemplated such as, for example, drive rods, drive cables, drive screws, etc.

Referring still to FIG. 1, the drive assembly may be tuned to provide a jaw clamping force, or jaw clamping force within a jaw clamping force range, to tissue clamped between jaw member 164 and blade 162, such as described in U.S. patent application Ser. No. 17/071,263, filed on Oct. 15, 2020, the entire contents of which are hereby incorporated herein by reference. Alternatively, the drive assembly may include a force limiting feature, e.g., a spring, whereby the clamping force applied to tissue clamped between jaw member 164 and blade 162 is limited to a particular jaw clamping force or a jaw clamping force within a jaw clamping force range, such as described in U.S. Pat. No. 10,368,898, issued on Aug. 6, 2019, the entire contents of which are hereby incorporated herein by reference.

One or more sensors 132 are provided to sense that clamp lever 130 has been actuated at least to the point of sufficient actuation and, thus, to sense whether clamping force is applied to tissue clamped between jaw member 164 and blade 162.

Continuing with reference to FIG. 1, waveguide 154, as noted above, extends from handle assembly 110 through inner sleeve 153 (FIG. 4). Waveguide 154 includes blade 162 disposed at a distal end thereof. Blade 162 may be integrally formed with waveguide 154 separately formed and subsequently attached (permanently or removably) to waveguide 154, or otherwise operably coupled with waveguide 154. Waveguide 154 and/or blade 162 may be formed from titanium, a titanium alloy, or other suitable electrically conductive material(s), although non-conductive materials are also contemplated. Waveguide 154 includes a proximal connector (not shown), e.g., a threaded male connector, configured for engagement, e.g., threaded engagement within a threaded female receiver, of ultrasonic transducer 140 such that ultrasonic motion produced by ultrasonic transducer 140 is transmitted along waveguide 154 to blade 162 for treating tissue clamped between blade 162 and jaw member 164 or positioned adjacent to blade 162.

Cable assembly 190 of surgical instrument 100 includes a cable 192, an ultrasonic plug 194, and an electrosurgical plug 196. Ultrasonic plug 194 is configured for connection with ultrasonic plug port 230 of surgical generator 200 while electrosurgical plug 196 is configured for connection with bipolar electrosurgical plug port 240 of surgical generator 200 and/or active monopolar electrosurgical plug port 250 of surgical generator 200. In configurations where generator 200 includes a common port, cable assembly 190 may include a common plug (not shown) configured to act as both the ultrasonic plug 194 and the electrosurgical plug 196.

Plural first electrical lead wires 197 electrically coupled to ultrasonic plug 194 extend through cable 192 and into handle assembly 110 for electrical connection to ultrasonic transducer 140 and/or activation button 120 to enable the selective supply of ultrasonic drive signals from surgical generator 200 to ultrasonic transducer 140 upon activation of ultrasonic energy. In addition, plural second electrical lead wires 199 are electrically coupled to electrosurgical plug 196 and extend through cable 192 into handle assembly 110. In bipolar configurations, separate second electrical lead wires 199 are electrically coupled to waveguide 510 and jaw member 164 (and/or different portions of jaw member 164) such that bipolar electrosurgical energy may be conducted between blade 162 and jaw member 164 (and/or between different portions of jaw member 164). In monopolar configurations, a second electrical lead wire 199 is electrically coupled to waveguide 154 such that monopolar electrosurgical energy may be supplied to tissue from blade 162. Alternatively or additionally, a second electrical lead wire 199 may electrically couple to jaw member 164 in the monopolar configuration to enable monopolar electrosurgical energy to be supplied to tissue from jaw member 164. In configurations where both bipolar and monopolar functionality are enabled, one or more of the second electrical lead wires 199 may be used for both the delivery of bipolar energy and monopolar energy; alternatively, bipolar and monopolar energy delivery may be provided by separate second electrical lead wires 199. One or more other second electrical lead wires 199 is electrically coupled to activation button 120 to enable the selective supply of electrosurgical energy from surgical generator 200 to waveguide 510 and/or jaw member 164 upon activation of electrosurgical energy.

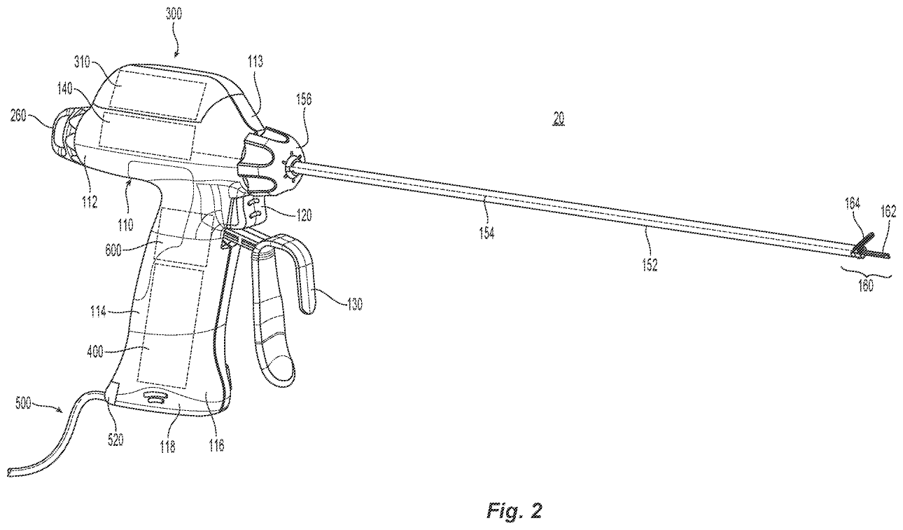

As an alternative to a remote generator 200, surgical system 10 may be at least partially cordless in that it incorporates an ultrasonic generator, an electrosurgical generator, and/or a power source, e.g., a battery, thereon or therein. In this manner, the connections from surgical instrument 100 to external devices, e.g., generator(s) and/or power source(s), is reduced or eliminated. More specifically, with reference to FIG. 2, another surgical system in accordance with the present disclosure is shown illustrated as a surgical instrument 20 supporting an ultrasonic generator 310, a power source (e.g., battery assembly 400), and an electrosurgical generator 600 thereon or therein. Surgical instrument 20 is similar to surgical instrument 100 (FIG. 1) and may include any of the features thereof except as explicitly contradicted below. Accordingly, only differences between surgical instrument 20 and surgical instrument 100 (FIG. 1) are described in detail below while similarities are omitted or summarily described.

Housing 112 of surgical instrument 20 includes a body portion 113 and a fixed handle portion 114 depending from body portion 113. Body portion 113 of housing 112 is configured to support an ultrasonic transducer and generator assembly (“TAG”) 300 including ultrasonic generator 310 and ultrasonic transducer 140. TAG 300 may be permanently engaged with body portion 113 of housing 112 or removable therefrom.

Fixed handle portion 114 of housing 112 defines a compartment 116 configured to receive battery assembly 400 and electrosurgical generator 600 and a door 118 configured to enclose compartment 116. An electrical connection assembly (not shown) is disposed within housing 112 and serves to electrically couple activation button 120, ultrasonic generator 310 of TAG 300, and battery assembly 400 with one another when TAG 300 is supported on or in body portion 113 of housing 112 and battery assembly 400 is disposed within compartment 116 of fixed handle portion 114 of housing 112, thus enabling activation of surgical instrument 20 in an ultrasonic mode in response to appropriate actuation of activation button 120. Further, the electrical connection assembly or a different electrical connection assembly disposed within housing 112 serves to electrically couple activation button 120, electrosurgical generator 600, battery assembly 400, and end effector assembly 160 (e.g., blade 162 and jaw member 164 and/or different portions of jaw member 164) with one another when electrosurgical generator 600 and battery assembly 400 are disposed within compartment 116 of fixed handle portion 114 of housing 112, thus enabling activation of surgical instrument 20 to supply electrosurgical energy, e.g., bipolar RF energy, in response to appropriate actuation of activation button 120. To enable the supply of monopolar electrosurgical energy, plug 720 of return electrode device 700 (FIG. 1) may be configured to connect to surgical instrument 20 (electrosurgical generator 600 thereof, more specifically), to complete a monopolar circuit through tissue and between surgical instrument 20 (e.g., blade 500 and/or jaw member 164) and return electrode device 700.

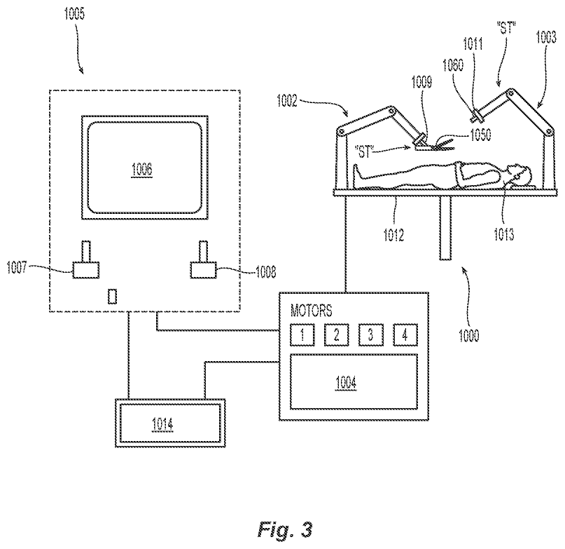

Turning to FIG. 3, a robotic surgical system in accordance with the aspects and features of the present disclosure is shown generally identified by reference numeral 1000. For the purposes herein, robotic surgical system 1000 is generally described. Aspects and features of robotic surgical system 1000 not germane to the understanding of the present disclosure are omitted to avoid obscuring the aspects and features of the present disclosure in unnecessary detail.

Robotic surgical system 1000 generally includes a plurality of robot arms 1002, 1003; a control device 1004; and an operating console 1005 coupled with control device 1004. Operating console 1005 may include a display device 1006, which may be set up in particular to display three dimensional images; and manual input devices 1007, 1008, by means of which a person (not shown), for example a surgeon, may be able to telemanipulate robot arms 1002, 1003 in a first operating mode. Robotic surgical system 1000 may be configured for use on a patient 1013 lying on a patient table 1012 to be treated in a minimally invasive manner. Robotic surgical system 1000 may further include a database 1014, in particular coupled to control device 1004, in which are stored, for example, pre-operative data from patient 1013 and/or anatomical atlases.

Each of the robot arms 1002, 1003 may include a plurality of members, which are connected through joints, and an attaching device 1009, 1011, to which may be attached, for example, a surgical tool “ST” supporting an end effector 1050, 1060. One of the surgical tools “ST” may be surgical instrument 100 (FIG. 1), surgical instrument 20 (FIG. 2), or any other suitable surgical instrument 20 configured for use in both an ultrasonic mode and one or more electrosurgical (bipolar and/or monopolar) modes, wherein manual actuation features, e.g., actuation button 120 (FIG. 1), clamp lever 130 (FIG. 1), etc., are replaced with robotic inputs. Further, sensor(s) for sensing the robotic inputs similarly as detailed above with respect to sensing clamp lever 130 (FIG. 1) may be provided. Robotic surgical system 1000 may include or be configured to connect to an ultrasonic generator, an electrosurgical generator, and/or a power source. The other surgical tool “ST” may include any other suitable surgical instrument, e.g., an endoscopic camera, other surgical tool, etc. Robot arms 1002, 1003 may be driven by electric drives, e.g., motors, that are connected to control device 1004. Control device 1004 (e.g., a computer) may be configured to activate the motors, in particular by means of a computer program, in such a way that robot arms 1002, 1003, their attaching devices 1009, 1011, and, thus, the surgical tools “ST” execute a desired movement and/or function according to a corresponding input from manual input devices 1007, 1008, respectively. Control device 1004 may also be configured in such a way that it regulates the movement of robot arms 1002, 1003 and/or of the motors.

Referring to FIG. 5, one embodiment of the present disclosure includes a combination ultrasonic and electrosurgical scalpel for use with treating tissue and is generally identified as reference numeral 600. Scalpel 600 is schematically shown for simplicity but is intended to include similar components to the instruments described above. In general, scalpel 600 includes a housing 112 which includes a transducer 140 disposed therein that operably couples to a waveguide 510 having a blade 500 disposed at a distal end thereof. The transducer 140 electrically couples to a switch 120 for transferring ultrasonic energy through the blade 500 upon activation thereof. The blade 500 also electrically connects to a switch 125 which provides monopolar energy to the blade 500 which is returned through a return path as detailed above (via an opposing jaw 164 or return pad 710—See FIG. 1). A single switch, e.g., switch 120, may be utilized to supply both energy modalities simultaneously or according to a mathematical algorithm.

In general, blades using highly directed monopolar energy to treat or dissect tissue are commonly referred to as “plasma blades”. Some tissues cut better than other tissues under this energy modality. An ultrasonic scalpel uses kinetic energy to directly heat and ablate tissue but cuts tissue in a much slower fashion and is typically not as precise depending on tissue type, pressure and velocity. For example, an ultrasonic scalpel may be used to deftly skeletonize vascular tissue in the liver or aggressively cut through thick avascular tissue, such as tendon and bone. Both ultrasonic scalpels and monopolar scalpels/pencils can be used for spot coagulation of small vasculature. A monopolar scalpel/pencil uses a special energy mode for coagulating tissue, but eschar may build up along the cutting edge in the process which may make the instrument less effective over prolonged use or require repeated cleaning during use thereof making the instrument less efficient. An ultrasonic scalpel can also spot coagulate by using a blunt surface on the blade or the side of the blade. Although an ultrasonic scalpel typically does not cut tissue as fast as a monopolar scalpel/pencil, an ultrasonic blade has minimal-to-no eschar buildup which may be more desirable for surgical purposes.

Referring again to FIGS. 5, and 6A-6C, blade 500 is shown and includes proximal, middle and distal edge portions, 500c, 500b, 500a, respectively, configured for tissue dissection by using either or both ultrasonic and or electrosurgical energy and proximal, middle and distal side portions 501c, 501b, 501a, respectively, configured for spot coagulation by using either or both ultrasonic and or electrosurgical energy. FIG. 5 shows the scalpel 600 in use in situ wherein the unique shape of the scalpel 600 together with the ability to control two different types of energy modalities for simultaneous or independent application allows the surgeon to more easily skeletonize larger vessels from the surrounding tissue as explained in more detail below. The blade 500 extends distally from the waveguide 510 disposed at a proximal end thereof which is disposed within housing 112 (FIG. 5). The shape of the blade 500 is tapered both vertically and horizontally (multi-axis tapered design) to form an elongated V-shaped tapered cutting edge 505 extending distally along a top surface thereof to the distal edge portion 500a and an elongated tapered thin edge extending along a side surface thereof to a flat spatula-like surface 507 at the distal side portion 501a. This blade shape enables the surgeon to utilize both energy modalities simultaneously or independently to enhance tissue treatment as explained in more detailed below.

Hence shaping blade 500 with a multi-axis tapered design (tapered along both vertical and horizontal axes) having dual energy modalities to combine the treatment capabilities of both an ultrasonic scalpel with a plasma-style monopolar pencil to enable quick dissection of tissue, regardless of tissue composition, and better spot coagulation without eschar build-up enables a surgeon to treat a multitude of tissue types in a precise and expeditious manner with little or no eschar build-up.

For example, as shown in FIGS. 6A and 6B, the blade 500 design enables the surgeon to enhance performance by combining both energy modalities. The elongated V-shaped tapered cutting edge 505 is coated with a highly conductive material 502 and extends from the proximal-to-distal end portions 500c-500a thereof and is electrically coupled to energy source 200 (with the return provided via REM). A flat, spatula-like, spot coagulation surface 507 is disposed on distal side portion 501a (actually dual-sided (not shown)) and is coated with an insulative material 503 (e.g., glass, ceramic, polymer or other material) that extends proximally along side portions 501b and 501a. The waveguide 510 is also coated with the same or similar insulative material 503 (e.g., glass, ceramic, polymer or other material).

The blade 500 is coated or covered by the insulating material 503 so that only the wanted area of treatment on the blade 500 and the place of electrical connection in the handset is exposed and may transfer energy. This insulation 503 may be in the form of an oxide layer, ceramic, glass, or other material directly coating the waveguide 510 and becoming integral to the waveguide 510. Further insulation may also be in the form of a covering such as a plastic sheath 900 or some other part of the handset.

Designing the blade 500 in this fashion enables the surgeon to energize both modalities simultaneously and treat various tissue types by simply manipulating the orientation of the scalpel 600. For example, if the surgeon wishes to dissect tissue the surgeon would orient the blade 500 such that the cutting edge 505 is in contact with the tissue to be dissected and energize the switch 120 which, in this case, energizes both modalities, monopolar high energy plasma and ultrasonic energy. In this instance, the plasma energy would dominate the effect on tissue and quickly dissect along the cutting edge 505. Similarly, for trying to use monopolar energy for spot coagulation purposes utilizing flat spatula surface 507, adding ultrasonic energy will be more effective than just the monopolar high energy plasma on the tissue along surface 507.

Or, alternatively, the surgeon could independently energize each modality and treat tissue with a specific part of the blade 500 particularly suited for that tissue type in a more efficient and expeditious manner, i.e., the highly concentrated electrical path on the cutting edge 505 to create a plasma-like cutting surface that will easily cut through conductive tissue and ultrasonic energy on the flat spatula surface 507 to handle the thicker, non-conductive tissues.

FIG. 6C shows an example of another embodiment of the present disclosure wherein the blade 500 is coupled to a switch 535 enabling different parts of the blade 500 to be energized for different purposes and/or to address safety concerns. For example, blade 500 is similar in all respects to the embodiments described above in FIGS. 6A and 6B with the exception that an insulative sheath 900 is disposed over the middle portion of the blade 500, e.g., middle edge portion 500b and middle side portion 501b. A leaf spring or other type of contact 525a operably couples to an electrical lead 530a on one end thereof and operably couples to the proximal edge portion 500c on the opposite end thereof. Likewise, a leaf spring or other type of contact 525b operably couples to an electrical lead 530b on one end thereof and operably couples to the proximal side portion 501c on the opposite end thereof. Electrical leads 530a and 530b are disposed on opposing ends of toggle switch 535 controlled by the user or an algorithm.

During use, toggle switch 535 determines which surface is energized for use while insulative sheath 900 electrically insulates the other area from electrical activation. For example, if the surgeon wants to dissect tissue along distal edge 500a, the surgeon activates toggle switch 530a which, in turn, activates leaf spring 525a to energize edges 500c, 500b and 500a. Sheath prevents electrical current from energizing surfaces 501a, 501b and 501c as a safety measure to avoid accidental treatment or collateral damage to tissue. Insulation 503, e.g., oxide layer, ceramic, glass, or other material (as mentioned above) coated or otherwise deposited on the waveguide 510, prevents surfaces 500 and 501 from shorting. Open circuit 530b (or 530a) keeps the non-active surface from being energized when not selected by toggle switch 535.

Generator 200 and/or an algorithm may determine the modality(s) of energy to be applied and/or select the parameters of energy delivery, combinations or power levels thereof (e.g., high versus low power of either or both monopolar and/or ultrasonic energy while activated). For example, in aspects, monopolar energy may be activated in a high power, dissection mode and ultrasonic energy may be activated in a low power mode for spot coagulation. In other aspects, monopolar energy may be deactivated while ultrasonic energy may be activated in a high power mode depending upon a particular surgical purpose. In aspects, the surgeon may manually control the combination of the energy modalities according to the surgeon's particular surgical preferences.

While several aspects of the disclosure have been detailed above and are shown in the drawings, it is not intended that the disclosure be limited thereto, as it is intended that the disclosure be as broad in scope as the art will allow and that the specification be read likewise. Therefore, the above description and accompanying drawings should not be construed as limiting, but merely as exemplifications of particular aspects. Those skilled in the art will envision other modifications within the scope and spirit of the claims appended hereto.

What is claimed is:

1. A combination ultrasonic and electrosurgical surgical instrument, comprising:
a housing;
an ultrasonic transducer disposed within the housing; and
a wave guide configured to support an ultrasonic blade operably coupled to the ultrasonic transducer, the ultrasonic blade configured to receive ultrasonic energy produced by the ultrasonic transducer, the ultrasonic blade tapered along both a vertical and a horizontal axis defined therealong and defining an elongated edge on an upper surface thereof terminating at a spatula-like distal end, wherein the elongated edge and side of the spatula-like distal end are coated with an electrically conductive material and are both adapted to connect to a source of electrosurgical energy such that activation of the electrosurgical energy source and the ultrasonic transducer treat tissue with both electrosurgical energy and ultrasonic energy.

2. The combination ultrasonic and electrosurgical surgical instrument according to claim 1, wherein the ultrasonic transducer is separably activable relative to the source of electrosurgical energy.

3. The combination ultrasonic and electrosurgical surgical instrument according to claim 1, wherein the ultrasonic transducer is energized by the source of electrosurgical energy.

4. The combination ultrasonic and electrosurgical surgical instrument according to claim 1, wherein the electrically conductive material on the elongated edge and the side of the spatula-like distal end are separated by an insulated material and are independently activatable by a switch.

5. The combination ultrasonic and electrosurgical surgical instrument according to claim 4, wherein an insulating sheath is disposed between the electrically conductive material on the elongated edge and the side of the spatula-like distal end.

6. The combination ultrasonic and electrosurgical surgical instrument according to claim 1, wherein both ultrasonic and monopolar energy are simultaneously provided to the elongated edge to quickly and finely dissect tissue.

7. The combination ultrasonic and electrosurgical surgical instrument according to claim 1, wherein monopolar energy is provided to the elongated edge to dissect tissue.

8. The combination ultrasonic and electrosurgical surgical instrument according to claim 1, wherein the side surface of the spatula-like distal end is used to simultaneously treat tissue with ultrasonic energy and monopolar energy to coagulate tissue.

9. The combination ultrasonic and electrosurgical surgical instrument according to claim 1, wherein the side surface of the spatula-like distal end is used to treat tissue with ultrasonic energy to coagulate tissue.

10. The combination ultrasonic and electrosurgical surgical instrument according to claim 1, wherein the waveguide is coated with an insulative material selected from the group consisting of glass, ceramic and polymer.

11. A combination ultrasonic and electrosurgical surgical instrument, comprising:
a housing;
an ultrasonic transducer disposed within the housing; and
a wave guide configured to support an ultrasonic blade operably coupled to the ultrasonic transducer, the ultrasonic blade configured to receive ultrasonic energy produced by the ultrasonic transducer, the ultrasonic blade tapered along both a vertical and a horizontal axis defined therealong and defining an elongated edge on an upper surface thereof terminating at a spatula-like distal end, wherein the elongated edge and side of the spatula-like distal end electrically conductive and separated by an insulative material, the elongated edge and spatula-like distal end adapted to independently connect to a source of electrosurgical energy such that activation of the electrosurgical energy source treats tissue with both monopolar electrosurgical energy and ultrasonic energy.

12. The combination ultrasonic and electrosurgical surgical instrument according to claim 11, wherein the ultrasonic transducer is adapted to connect to a separate source of electrical energy.

13. The combination ultrasonic and electrosurgical surgical instrument according to claim 11, wherein the electrically conductive material on the elongated edge and the side of the spatula-like distal end are independently activatable by a switch.

14. The combination ultrasonic and electrosurgical surgical instrument according to claim 11, wherein the electrically conductive material on the elongated edge and the side of the spatula-like distal end are independently activatable by a switch controlled by an algorithm.

15. The combination ultrasonic and electrosurgical surgical instrument according to claim 11, wherein an insulating sheath is disposed between the electrically conductive material on the elongated edge and the side of the spatula-like distal end.

16. The combination ultrasonic and electrosurgical surgical instrument according to claim 11, wherein both ultrasonic and monopolar energy are simultaneously provided to the elongated edge to quickly and finely dissect tissue.

17. The combination ultrasonic and electrosurgical surgical instrument according to claim 11, wherein monopolar energy is provided to the elongated edge to dissect tissue.

18. The combination ultrasonic and electrosurgical surgical instrument according to claim 11, wherein the side surface of the spatula-like distal end is used to simultaneously treat tissue with ultrasonic energy and monopolar energy to coagulate tissue.

19. The combination ultrasonic and electrosurgical surgical instrument according to claim 11, wherein the side surface of the spatula-like distal end is used to treat tissue with ultrasonic energy to coagulate tissue.

20. The combination ultrasonic and electrosurgical surgical instrument according to claim 11, wherein the waveguide is coated with an insulative material selected from the group consisting of glass, ceramic and polymer.
